(19) United States
(12) Patent Application Publication
(10) Pub. No.: US 2012/0115871 A1
(43) Pub. Date: May 10, 2012

(54) METHOD OF TREATMENT OF DEPRESSION

(75) Inventor: Christopher D. Breder, Bethesda, MD (US)

(73) Assignee: Supemus Pharmaceuticals, Inc

(21) Appl. No.: 13/318,007

(22) PCT Filed: Apr. 29, 2010
(86) PCT No.: PCT/US10/32974

§ 371 Date: Jan. 20, 2012

Related U.S. Application Data
(60) Provisional application No. 61/174,084, filed on Apr. 30, 2009.

(51) Int. Cl. A61K 31/5375; A61P 25/24

(52) U.S. Cl. 514/239.2

(57) ABSTRACT

The invention comprises a method of treatment of depression or depression-related disorders by a pharmaceutical agent exhibiting combined serotonergic or noradrenergic reuptake transporters and monoamine receptor activity.

### CROSS-REFERENCE TO RELATED APPLICATIONS

This application claims priority to U.S. Provisional Application No. 61/174,084, filed Apr. 30, 2009, the disclosure of which is incorporated herein by reference in its entirety.

### BACKGROUND

Viloxazine (Emovit®, Vivalan®, Vivarint®, Vicilan®) is a bicyclic antidepressant morpholine derivative that inhibits the reuptake of norepinephrine. It is a racemic compound with two isomers, the S(−)-isomer being five times more pharmacologically active than the R(+)-isomer. Viloxazine hydrochloride has been approved in Italy, Belgium, the United States, England, Ireland, Germany, Portugal, Spain, the former Yugoslavia, France, Slovakia, for the treatment of clinical depression.

The principle pharmacology of viloxazine is believed to be the inhibition of noradrenergic reuptake transporters (155 nM) with very weak activity at the serotonin reuptake inhibitor (17.3 μm) (Tatsumi et al [1997] Eur J Pharmacol 340 (2-3): 249-58). The dose in adults varies from 150 to 800 mg per day. However, unlike the tricyclic antidepressants, viloxazine does not have marked antimuscarinic, cardiotoxic, or sedative properties. Side effects of viloxazine include nausea, vomiting, insomnia, loss of appetite, increased erythrocyte sedimentation, EKG and EEG anomalies, epigastric pain, diarrhea, constipation, vertigo, orthostatic hypotension, edema of the lower extremities, dysarthria, tremor, psychomotor agitation, mental confusion, inappropriate secretion of antidiuretic hormone, increased transaminases, seizure, and increased libido (Chebili S, Abaoub A, Mezouane B, Le Goff J F (1998). “Antidepressants and sexual stimulation: the correlation” L'Encephale 24 (3): 180-4.)

The current invention discloses a method of treatment of depression with viloxazine that enhances clinical response while minimizing incidence and severity of side effects.

### SUMMARY OF THE INVENTION

The invention provides a method of identifying compounds for the treatment of depression and/or similar disorders, comprising (1) selecting one or a combination of active agents with known activity inhibiting either serotonin or noradrenergic reuptake transporters; (2) conducting a receptor screening assay on the selected agent(s) to identify activity on at least one dopaminergic, serotonergic or gabaergic receptor where the activity is known to inhibit depression or where the opposite activity is associated with depression (e.g., where a compound is determined to be a receptor antagonist and if stimulation (agonism) of that receptor is associated with the onset or worsening of depression); (3) determining if said activity is agonistic or antagonistic; (4) selecting among the screened active agents at least one that targets the most diverse types of depression-associated receptors; and (5) optimizing the total dosage of the selected active agent(s).

Identification of monoamine agonist/antagonist activity in agents that inhibit serotonin or noradrenergic reuptake is important, since it allows for selection of drugs that have more than one therapeutic target (e.g. both 5HT7 and noradrenergic reuptake transporter). This is superior to taking a combination of therapies to achieve multiple targets because of the enhanced patient compliance with the reduced pill load. It also can lead to a lower dose, since different receptor activities may be additive, or even synergistic, in their effect.

Use of molecules that target a specific class of receptors with a limited distribution in the brain is also potentially beneficial in that it limits the potential for side effects. This restricted set of neural pathways is less likely to have “off-target” effect in the systems not involved in the desired activity.

In one embodiment, the invention comprises a method of treatment of depression and depression-related disorders including, but not limited, to mood disorders such as bipolar disorder or disorders where depression may be a co-morbid syndrome, including but not limited to, fibromyalgia, by a pharmaceutical agent exhibiting both serotonin or noradrenergic reuptake activity and 5-HT7 antagonistic activity.

In another embodiment, the invention provides a method of treatment of depression and depression related disorders by a pharmaceutical agent exhibiting combined serotonin or noradrenergic reuptake activity and 5-HT7 antagonistic activity, wherein the total dosage of the pharmaceutical agent is smaller than the dosage anticipated on the premise of the serotonin or noradrenergic reuptake activity only.

In yet another embodiment of the invention, the pharmaceutical agent is viloxazine.

### BRIEF DESCRIPTION OF THE DRAWINGS

FIG. 1 shows a competition curve obtained with compound SPN-809V with human 5-HT7 receptor.

FIG. 2 shows the agonist effect of the compound SPN-809V with human 5HT7 receptor.

FIG. 3 shows the antagonist effect of the compound SPN-809V with human 5HT7 receptor.

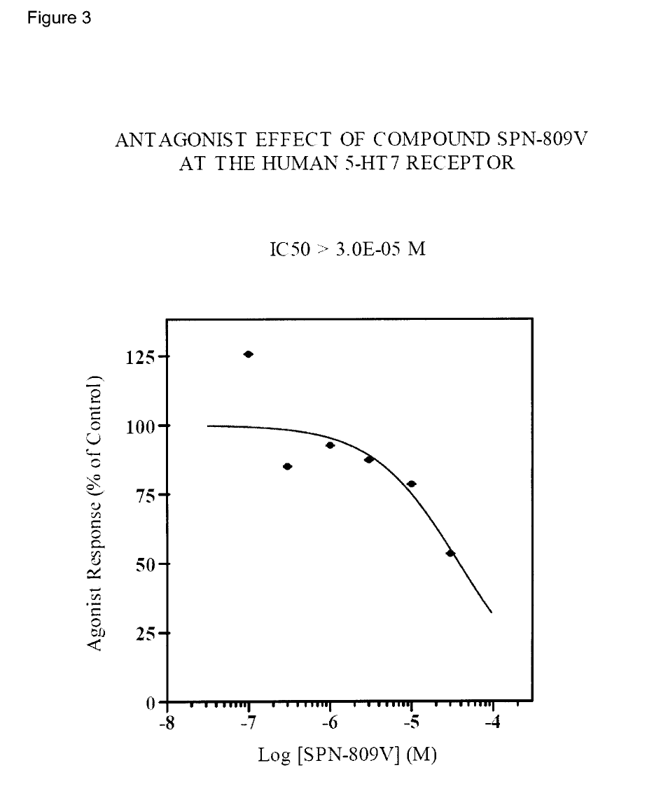

### DETAILED DESCRIPTION OF THE INVENTION

Unless otherwise specified, “a” or “an” means “one or more.” Notwithstanding the well-documented conventional understanding in the art that viloxazine exerts its pharmaceutical activity by inhibiting noradrenergic reuptake, the present invention is based on the unexpected discovery that viloxazine also has specific antagonist activity at the 5-HT7 (serotonin) receptor. Without being held to or bound by theory, the present invention is thought to take advantage of this discovery. In one embodiment, for example, the present invention provides a method of treating depression using a dose of viloxazine that is substantially below what is currently deemed therapeutic. Significantly, lower daily dosing of the viloxazine can result in the diminishing frequency and severity, if at all, of the adverse effects commonly associated with the treatment of depression using viloxazine.

In a preferred embodiment, the invention provides a method of treatment of depression or depression-related disorders in human subjects by administering viloxazine in the total daily dose that is at least 10% lower than the current minimally effective dose of 2.14 mg/kg. In other embodiments, the dose is 15% lower, 25% lower, 35% lower, and 50% lower than the current dose. Dosage ranges of 1.1 mg/kg/day to 9.7 mg/kg/day or approximately 20 to 800 mg for pediatric (aged 6 to 17) and adult population are also provided.

According to the invention, viloxazine can be administered in the amount of from 100 to 600 mg/day. In another embodiment, the daily dose of viloxazine constitutes from 150 to 400 mg/day. In yet further embodiment of the invention, viloxazine is administered in the amount of up to 300 mg/day.

In another embodiment, the invention encompasses a method of treatment of depression or depression-related disorders with viloxazine that is characterized by an improved adverse effect profile. The adverse effects that are diminished by the method of the present invention include, but are not limited to, nausea, vomiting, insomnia, loss of appetite, increased erythrocyte sedimentation, EKG and EEG anomalies, epigastric pain, diarrhea, constipation, vertigo, orthostatic hypotension, edema of the lower extremities, dysarthria, tremor, psychomotor agitation, mental confusion, inappropriate secretion of antidiuretic hormone, increased transaminases, seizure, and increased libido. Hence, the inventive method provides for the treatment of depression without, or at least with far less frequency than with conventional viloxazine-treatment, one, two, six or more of these listed side effects. The efficacy and the adverse effect profile of the lower dose treatment of the current invention are evaluated in a randomized, placebo controlled trial.

### Viloxazine Activity on 5-HT Receptors

A heterologous competition assay was to determine the relative affinity of viloxazine for 5-HT receptors. Briefly, recombinant 5-HT7 receptors were expressed in a CHO cell line. The receptors were then saturated with a tritiated receptor-specific ligand at concentrations known to be saturating. Thereupon, 10 μm viloxazine was added to the cells in the presence of non-specific ligand and incubated. In this way, viloxazine is allowed to “compete” with the receptor-specific ligand, such that greater displacement (i.e., % inhibition) is indicative to greater binding strength of viloxazine at a given receptor. “Specific binding” refers here to the difference in the binding of the ligand to the receptors in the presence and absence of an excess of the viloxazine. The conditions and results of the assay are summarized in the Table 1.

TABLE 1
Conditions of the displacement assay at select serotonin receptors for viloxazine

| | | | | | | % | |
|---|---|---|---|---|---|---|---|
| Receptor | Ligand | Conc. | | Non-specific | Incubation | Inhib. | Detection method |
| 5-HT_{1A} (h) | [³H]8-OH-DPAT | 0.3 | nM | 8-OH-DPAT (10 μM) | 60 min/22° C. | 66 | Scintillation counting |
| 5-HT_{1B} | [¹²⁵I]CYP | 0.1 | nM | serotonin (10 μM) | 120 min/37° C. | 78 | Scintillation counting |
| | (+30 μM | | | | | | |
| | (−)propranolol) | | | | | | |
| 5-HT_{1D} | [³H]serotonin | 1 | nM | serotonin (10 μM) | 60 min/22° C. | 18 | Scintillation counting |
| 5-HT_{2A} (h) | [³H]ketanserin | 0.5 | nM | ketanserin (1 μM) | 60 min/22° C. | −17* | Scintillation counting |
| 5-HT_{2C} (h) | [³H]mesulergine | 1 | nM | RS-102221 (10 μM) | 60 min/37° C. | 56 | Scintillation counting |
| 5-HT₃ (h) | [³H]BRL 43694 | 0.5 | nM | MDL 72222 (10 μM) | 120 min/22° C. | 18 | Scintillation counting |
| 5-HT₄ₑ (h) | [³H]GR 113808 | 0.3 | nM | serotonin (100 μM) | 60 min/37° C. | 16 | Scintillation counting |
| 5-HT_{5A} (h) | [³H]LSD | 1 | nM | serotonin (100 μM) | 60 min/37° C. | 15 | Scintillation counting |
| 5-HT₆ (h) | [³H]LSD | 2 | nM | serotonin (100 μM) | 120 min/37° C. | 6 | Scintillation counting |
| 5-HT₇ (h) | [³H]LSD | 4 | nM | serotonin (10 μM) | 120 min/22° C. | 70 | Scintillation counting |

*A negative number reflects 0% inhibition. Since % inhibition equals (100 minus measured specific binding in the presence of SPN809V)/control specific binding), a negative number represents a condition where the binding of the radioactive test ligand was greater in the presence of SPN-809V. This reflects either the variability in the radioactive control ligand binding or facilitation by the test ligand.

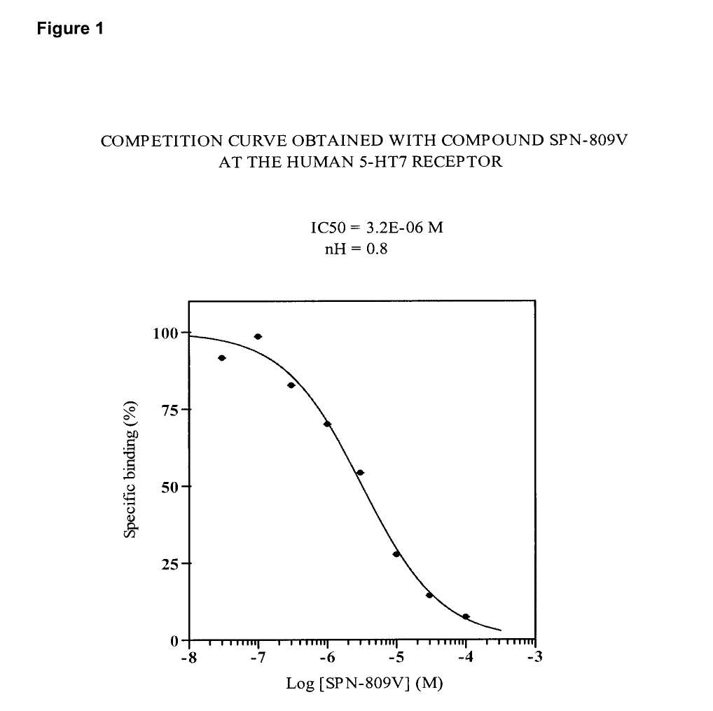

The affinity of viloxazine for 5-HT7 receptors was further characterized by determining the IC50 (i.e., the concentration of viloxazine that can inhibit 50% of control specific binding). For this experiment, a range of viloxazine concentrations was selected for the ligand blocking assay. The IC50 was determined using non-linear regression analysis of the competition curves using Hill equation curve fitting. The inhibition constants Ki were calculated using Cheng Prusoff equation. Ki is defined as the concentration of the competing ligand (viloxazine) that bound to half the binding sites at equilibrium in the absence of radioligand or other competitors, The results of the affinity assay are summarized in Tables 2 and 3, and in FIG. 1.

TABLE 2
IC₅₀ Determination Data

| | | % of Control | | |
|---|---|---|---|---|
| | | Specific Binding | | |
| Receptor | Concentration (M) | 1st | 2nd | Mean |
| 5-HT_{1A} (h) | 3.0E−08 | 97.8 | 99.8 | 98.8 |
| | 1.0E−07 | 93.8 | 96.9 | 95.4 |
| | 3.0E−07 | 104.7 | 110.3 | 107.5 |
| | 1.0E−06 | 104.8 | 109.1 | 107.0 |
| | 3.0E−06 | 76.5 | 71.4 | 73.9 |
| | 1.0E−05 | 32.5 | 41.3 | 36.9 |
| | 3.0E−05 | 21.9 | 19.6 | 20.7 |
| | 1.0E−04 | 5.3 | 5.8 | 5.5 |
| 5-HT_{1B} | 3.0E−08 | 102.0 | 99.9 | 101.0 |
| | 1.0E−07 | 97.6 | 92.4 | 95.0 |
| | 3.0E−07 | 92.4 | 82.7 | 87.6 |
| | 1.0E−06 | 77.7 | 79.0 | 78.4 |
| | 3.0E−06 | 61.5 | 52.6 | 57.1 |
| | 1.0E−05 | 36.6 | 27.1 | 31.9 |
| | 3.0E−05 | 13.7 | 4.5 | 9.1 |
| | 1.0E−04 | −10.4 | −12.4 | −11.4 |
| 5-HT_{2C} (h) | 3.0E−08 | 97.9 | 125.8 | 111.9 |
| | 1.0E−07 | 116.6 | 111.5 | 114.0 |
| | 3.0E−07 | 92.9 | 102.7 | 97.8 |
| | 1.0E−06 | 108.2 | 104.2 | 106.2 |
| | 3.0E−06 | 90.6 | 91.9 | 91.3 |
| | 1.0E−05 | 61.6 | 63.1 | 62.3 |
| | 3.0E−05 | 33.1 | 36.6 | 34.8 |
| | 1.0E−04 | 8.4 | 14.3 | 11.4 |
| 5-HT₇ (h) | 3.0E−08 | 90.6 | 92.7 | 91.7 |
| | 1.0E−07 | 102.9 | 94.2 | 98.5 |
| | 3.0E−07 | 80.4 | 85.1 | 82.7 |
| | 1.0E−06 | 73.5 | 66.5 | 70.0 |
| | 3.0E−06 | 48.2 | 60.2 | 54.2 |
| | 1.0E−05 | 27.3 | 27.9 | 27.6 |
| | 3.0E−05 | 15.3 | 13.2 | 14.3 |
| | 1.0E−04 | 6.5 | 8.1 | 7.3 |

TABLE 3
Summary of IC50 determination at select serotonin receptors for
Viloxazine.

| | Reference | | | |
|---|---|---|---|---|
| Assay | compound | IC₅₀ (M) | Kᵢ (M) | n_{(H)} |
| 5-HT_{1A} (h) | 8-OH-DPAT | 7.1E−06 | 4.5E−06 | 1.3 |
| 5-HT_{1B} | serotonin | 3.8E−06 | 2.3E−06 | 1.0 |
| 5-HT_{2C} (h) | RS-102221 | 1.4E−05 | 6.4E−06 | 1.0 |
| 5-HT₇ (h) | serotonin | 3.2E−06 | 1.2E−06 | 0.8 |

The nature of the binding (i.e., agonist or antagonist) was next determined. Briefly, an assay was designed that examined the agonist effect on the 5HT7 receptor, i.e., the generation of cAMP or the blockade of this effect when stimulated by a 5HT7 agonist, serotonin. This was also done with a range of concentrations to determine the relative agonist versus antagonist binding Ki. The EC₅₀ values (concentration producing a half-maximal specific response) and IC₅₀ values (a concentration causing a half-maximal inhibition of the control-specific agonist response) were determined by a non-linear regression analysis of the concentration-response curves generated with mean replicate values using Hill equation curve fitting. The apparent dissociation constants for antagonists K_{b} were calculated using the modified Cheng Prusoff equation.

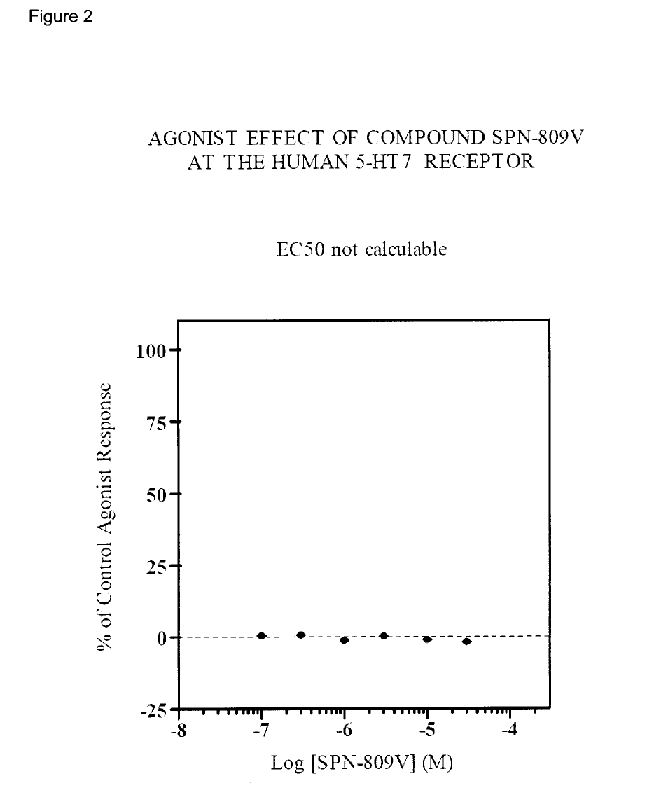

The conditions of the screening are represented in Table 4. Results of the functional assays are seen in FIGS. 2 (agonist assay) and 3 (antagonist assay). The agonist assay demonstrated no measurable response (FIG. 2). The antagonist assay yielded a weak response with an IC₅₀ greater than 3.0×10⁻⁵ M.

TABLE 4
Conditions for 5HT7 Functional Assay

| | Reference | Incubation | Reaction | Method of |
|---|---|---|---|---|
| Assay | compound | conditions | product | detection |
| 5-HT₇ (h) | none | 45 min/37° C. | cAMP | HTRF |
| (agonist effect) | | | | |
| 5-HT₇ (h) | serotonin | 45 min/37° C. | cAMP | HTRF |
| (antagonist effect) | | | | |

All of the publications, patent applications and patents cited in this specification, including the following references, are incorporated herein by reference in their entirety.
1. Vanhoenacker P, Haegeman G, Leysen J E. 5-HT7 receptors: current knowledge and future prospects. Trends Pharmacol Sci 2000; 21(2):70-7.
2. Lucchelli A, Santagostino-Barbone M G, D'Agostino G, Masoero E, Tonini M. The interaction of antidepressant drugs with enteric 5-HT7 receptors. Naunyn Schmiedebergs Arch Pharmacol 2000; 362(3):284-9.
3. Hedlund P B, Huitron-Resendiz S, Henriksen S J, Sutcliffe J G. 5-HT7 receptor inhibition and inactivation induce antidepressant like behavior and sleep pattern. Biol Psychiatry 2005; 58 (10):831-7.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

What is claimed is:

1. A method of treating depression or depression-related disorders in a patient, comprising administering to the patient a dose of viloxazine that less than about 1.95 mg/kg/day, wherein the treatment diminishes the frequency or severity of adverse effects of doses of viloxazine greater than about 1.95 mg/kg/day.

2. A method of treating depression or depression-related disorders comprising:
a. selecting several active agents with known activity inhibiting either serotonin or noradrenergic reuptake transporters;
b. conducting a receptor screening assay on these same agents to identify activity for at least one dopaminergic, serotonergic or gabaergic receptor where the activity is known to inhibit depression or opposite activity is associated with depression;
c. determining if said activity is agonistic or antagonistic in nature;
d. by the results of steps b) and c), choosing among the screened active agents at least one that targets the most diverse types of depression-associated receptors;
e. optimizing the total dosage of the active agent(s), taking into account results of steps b)-d).

3. A method of treating depression and/or or depression-related disorders by a pharmaceutical agent exhibiting combined serotonin or noradrenergic reuptake inhibition and 5-HT7 antagonistic activity, wherein the total dosage of the pharmaceutical agent is less than the dosage anticipated on the basis of only noradrenergic reuptake transporter activity.

4. The method of claim 2, wherein the pharmaceutical agent is viloxazine.

5. The method of claim 3, wherein the pharmaceutical agent is administered in a dose range of from 20 to 800 mg/day.

6. The method of claim 3, wherein the pharmaceutical agent is administered in a dose range of from 1.1 mg/kg/day to 9.7 mg/kg/day.

7. The method of claim 3, wherein the pharmaceutical agent has a dose that is 15% lower, 25% lower, 35% lower, or 50% lower than 2.14 mg/kg.

8. The method of claim 3, wherein there is a decrease in the side effects associated with viloxazine administration.

9. A pharmaceutical composition for the treatment depression or a depressive disorder in a patient comprising less than about 10 mg of viloxazine and a pharmaceutical carrier.
